(19) United States
(12) Patent Application Publication
(10) Pub. No.: US 2024/0415163 A1
(43) Pub. Date: Dec. 19, 2024

(54) FLAVOR BANDING OF CAPSULES

(71) Applicant: L. PERRIGO COMPANY, Allegan, MI (US)

(72) Inventors: Inderdeep BHATIA, Kalamazoo, MI (US); Amit BANSAL, Portage, MI (US); Kiran Kumar MUPPIREDDY, Portage, MI (US); Carlos PAZ, Fairview, NJ (US)

(21) Appl. No.: 18/210,977

(22) Filed: Jun. 16, 2023

(51) Int. Cl. A23P 10/30; A23L 27/00; A23L 27/30; A23L 29/281

(52) U.S. Cl. CPC A23P 10/30; A23L 27/34; A23L 27/72; A23L 29/284

(57) ABSTRACT

The present disclosure relates to a composition suitable for a flavored banding portion of a tamper-evidence, two-part capsule and to the tamper-evident, two-part capsule having a flavored banding portion. The composition suitable for the flavored capsule banding portion includes a gelling agent, a surfactant, and a flavor system including a sweetener and a flavor configured to provide a capsule band that is effective to provide tamper evidence to a two-part capsule.

### TECHNICAL FIELD

The present disclosure relates to a pharmaceutical capsule, and more specifically, a pharmaceutical capsule having a flavored band that joins a first capsule shell portion to a second capsule shell portion.

### BACKGROUND

Pharmaceutical capsules having a band that joins a first capsule shell portion and a second capsule shell portion are well known. The band is helpful to prevent misuse and to provide tamper evidence to the capsule. In some circumstances, however, some adults and/or children may have trouble taking such pharmaceutical capsules for one or more reasons. Given the lack of flavored capsule dosage form options for over the counter and prescriptions drugs in the current market, it has been proposed to include flavors and/or sweeteners in the capsule band to help increase the acceptance of the capsule. However, it has been discovered that adding flavors and/or sweeteners to the capsule band tends to compromise the tamper evidence capability of the capsule band.

### SUMMARY

In one approach or embodiment, the present disclosure relates to a composition suitable for a flavored banding portion of a tamper-evident, two-part capsule. In aspect of this embodiment, the composition includes at least about 50 weight percent, on a dry basis, of a gelling agent; a surfactant; and a flavor system including a sweetener and a flavor. In another aspect, a weight ratio of the flavor system to the surfactant is about 10:1 to about 25:1, and wherein the composition is effective to provide tamper evidence to a two-part capsule that complies with the requirements of 21 C.F.R. Section 211.132.

In another approach or embodiment, the composition suitable for a flavored banding portion of a tamper-evidence two-part capsule of the previous paragraph may include one or more optional feature or embodiments in any combination. These optional features or embodiments include one or more of the following: wherein a weight ratio of the gelling agent relative to the surfactant is about 20:1 to about 30:1, a weight ratio of the gelling agent to the sweetener is about 5:1 to about 30:1, and/or a weight ratio of the surfactant to the sweetener is about 0.2:1 to about 2:1; and/or wherein the gelling agent is selected form the group consisting of gelatin, locust bean gum, agar, guar, carrageenan, konjac, pectin, or combinations thereof; and/or wherein the composition includes, on a dry basis, about 50 to about 95 weight percent of the gelling agent; and/or wherein the surfactant is selected from the group consisting of polyoxyethylene sorbitan monooleate, sorbitan monostearate, sorbitan stearate, or combinations thereof; and/or wherein the composition includes, on a dry basis, about 1 to about 4 weight percent of the surfactant; and/or wherein the sweetener is a natural or non-nutritive sweetener selected from the group consisting of sugar, sucrose, glucose, sucralose, aspartame, acesulfame k, saccharin, stevia, or combinations thereof; and/or wherein the composition includes, on a dry basis, about 1 to about 50 weight percent of the sweetener; and/or wherein the weight ratio of the gelling agent to the sweetener is about 20:1 to about 25:1 and the weight ratio of the surfactant to the sweetener is about 0.8:1 to about 1.2:1; and/or wherein the gelling agent is gelatin, the surfactant is polyoxyethylene sorbitan monooleate, and the sweetener is sucralose; and/or wherein the composition includes, on a dry basis, up to about 50 weight percent of the flavor.

In yet a further approach or embodiment, the present disclosure also relates to a tamper-evident, two-part capsule having a flavored banding portion. In aspects of this further embodiment, the capsule has two capsule parts and a flavored banding portion encircling an interface between the two capsule parts effective to provide tamper evidence to the capsule. In other aspect, the flavored banding portion is provided by a composition as described by any embodiment of this Summary and at least including (i) at least about 50 weight percent, on a dry basis, of a gelling agent, (ii) a surfactant, and (iii) a flavor system including a sweetener and a flavor, and wherein a weight ratio of the flavor system to the surfactant is about 10:1 to about 25:1. So configured, the tamper-evident, two-part capsule is tamper evident pursuant to the requirements of 21 C.F.R. Section 211.132.

In yet other approaches or embodiments, the tamper-evident, two-part capsule of the previous paragraph may include one or more optional features or embodiments in any combination. These optional features or embodiments include one or more of the following: wherein a weight ratio of the gelling agent relative to the surfactant is about 20:1 to about 30:1, a weight ratio of the gelling agent to the sweetener is about 5:1 to about 30:1, and/or a weight ratio of the surfactant to the sweetener is about 0.2:1 to about 2:1; and/or wherein the gelling agent is selected form the group consisting of gelatin, locust bean gum, agar, guar, carrageenan, konjac, pectin, or combinations thereof; and/or wherein the composition includes, on a dry basis, about 50 to about 95 weight percent of the gelling agent; and/or wherein the composition includes, on a dry basis, about 50 to about 60 weight percent of the gelling agent; and/or wherein the surfactant is selected from the group consisting of polyoxyethylene sorbitan monooleate, sorbitan monostearate, sorbitan stearate, or combinations thereof; and/or wherein the composition includes, on a dry basis, about 1 to about 4 weight percent of the surfactant; and/or wherein the sweetener is a natural or non-nutritive sweetener selected from the group consisting of sugar, sucrose, glucose, sucralose, aspartame, acesulfame k, saccharin, stevia, or combinations thereof; and/or wherein the composition includes, on a dry basis, about 1 to about 50 weight percent of the sweetener; and/or wherein the gelling agent is gelatin, the surfactant is polyoxyethylene sorbitan monooleate, and the sweetener is sucralose; and/or wherein the composition includes, on a dry basis, up to about 50 weight percent of the flavor; and/or wherein the capsule has a size of 00 to 5 and about 2 mg to about 5 mg of the flavored banding portion per capsule; and/or wherein the capsule has a size 4 and includes about 2.8 to about 3.2 mg of the flavored banding portion per capsule; and/or wherein the capsule is a gelatin capsule and the gelling agent is gelatin.

In yet further embodiments, the use of any embodiment of the flavor banded compositions as described in this Summary is also described herein for providing a tamper evident two-part capsule pursuant to 21 C.F.R. Section 211.132.

### DETAILED DESCRIPTION

The present disclosure relates to a composition suitable for a flavored banding portion of a tamper-evidence, two-part capsule and to the tamper-evident, two-part capsule having a flavored banding portion. The composition suitable for the flavored capsule banding portion includes at least a gelling agent, a surfactant, and a flavor system including a sweetener and a suitable flavor configured to provide a capsule band that is effective to provide tamper evidence to a two-part capsule. The flavor band composition may optionally also include a colorant. As discussed more below, in order to provide tamper evidence when including the flavor system (e.g., sweetener and flavor) in the banding composition, it has been discovered that certain relationships of one or more of the gelling agent, the surfactant, the sweetener, and/or the flavor system were discovered in order to maintain tamper evidence of the capsules herein when including the flavor. As used herein, capsule tamper evidence is set forth in 21 C.F.R. Section 211.132.

In one approach or embodiment, the composition suitable for the flavored banding portion of the capsules herein includes at least one or more of the following unique compositional relationships of the gelatin, the surfactant, and/or the flavor system (e.g., a sweetener and a flavor component): (a) at least about 50 weight percent of a gelling agent, (b) a weight ratio of the gelling agent relative to the surfactant of about 20:1 to about 30:1, (c) a weight ratio of the gelling agent to the sweetener of about 5:1 to about 30:1, (d) a weight ratio of the surfactant to the sweetener of about 0.2:1 to about 2:1, and/or (e) a weight ratio of the flavor system to the surfactant of about 10:1 to about 25:1 to provide suitable tamper evidence in the context of a flavored banding capsule for a two-part capsule when including one or more flavorants in the banding composition. Each component and relationship will be described further.

The gelling agent of the flavor banding compositions herein may be any suitable gelling agent used in pharmaceutical capsules. In approaches or embodiments, the gelling agent may include, but is not limited to, gelatin, locust bean gum, agar, guar, carrageenan, konjac, pectin, or combinations thereof. Preferably, the gelling agent is selected for compatibility with the capsule, and most preferably, is gelatin. In embodiments, the compositions herein include, on a dry basis, about 50 to about 95 weight percent of the gelling agent in order to provide the tamper evidence, and more preferably, about 50 to about 60 weight percent of the gelling agent, and most preferably about 50 to about 58 weight percent of the gelling agent to provide tamper evidence.

The surfactant of the flavor banding compositions herein may also be any suitable surfactant used in pharmaceutical capsules. In approaches or embodiments, the surfactant may include, but is not limited to, polyoxyethylene sorbitan monooleate, sorbitan monostearate, sorbitan stearate, or combinations thereof. Preferably, the surfactant is polysorbate 80 or polyoxyethylene sorbitan monooleate. In some approaches, the composition may include, on a dry basis, about 1 to about 4 weight percent of the surfactant, more preferably, about 1.8 to about 3 weight percent of the surfactant, and most preferably about 2 to about 2.5 weight percent of the surfactant.

The flavor systems of the flavor banding compositions include a sweetener and a flavor component combined. The sweetener may be any natural or non-nutritive sweetener. For instance, the sweetener may be sugar, sucrose, glucose, sucralose, aspartame, acesulfame k, saccharin, stevia, or combinations thereof. Preferably, the sweetener is a non-nutritive sweetener, and most preferably sucralose. In some approaches, the total banding compositions herein may include, on a dry basis, about 1 to about 50 weight percent of the sweetener, in other approaches, about 1 to about 20 weight percent, about 1 to about 10 weight percent, or about 1 to about 5 weight percent of the sweetener. In context of the flavor system (e.g., the sweetener and flavor component combined), the sweetener may be about 4 to about 6 weight percent of the flavor system (dry basis), and in other approaches, about 4.2 to about 5.8 weight percent of the flavor system.

The flavor systems of the flavor banding compositions herein also include a flavor, a flavorant, or a flavoring agent, and as used herein, such terms are interchangeable. As used herein, a flavor or flavoring agent may be any ingredient, compound, or substance having a composition effective to provide a taste and/or smell and may include, for instance, any substance, compound, and/or ingredient configured to provide a sweet, sour, salty, bitter, and/or savory flavor. A flavor or flavoring agent may include a sweet flavoring agent or sweet compound, a sour flavoring agent or sour compound, a salty flavoring agent or salty compound, a bitter flavoring agent or bitter compound, and/or a savory flavoring agent or savory compound. The flavors or flavoring agents herein may be natural and/or artificial flavors and, in some approaches, may be any flavor, flavoring, component, compound, and/or ingredient as set forth in 21 C.F.R. Section 101.22. Suitable flavors may be, but not limited to, mint, orange, berry, citrus, vanilla, strawberry, or combinations thereof. The total banding compositions herein, in some embodiments, may include, on a dry basis, about 1 to about 50 weight percent of flavors or flavoring agents, about 10 to about 50 weight percent, about 20 to about 50 weight percent, about 30 to about 50 weight percent of the flavors or flavoring agents, and more preferably about 35 to about 45 weight percent of the flavors or flavoring agent. In context of the flavor system (e.g., the sweetener and flavor component combined), the flavor component may be, on a dry basis, about 94 to about 96 weight percent of the flavor system, and in other approaches, about 94.2 to about 95.8 weight percent of the flavor system.

Optionally, the compositions herein may include a color or colorant. When included, the total banding compositions herein may include, on a dry basis, about 0 to about 4 weight percent, about 0.5 to about 3 weight percent, about 0.8 to about 2 weight percent, or about 0.8 to about 1.5 weight percent of one or more suitable colorants.

As shown in the Examples below, it has been discovered that when the flavor systems herein (including the sweetener and the flavor) are included in the banding composition, in order to maintain tamper evidence, certain unique relationships need to be maintained between the gelling agent, the surfactant, the sweetener, and/or the flavor system for achieving tamper evidence per 21 CFR 211.132 when using the flavorants. For instance, when the banding compositions includes, on a dry basis, about 1 to about 50 weight percent flavors (or other ranges noted above), then one or more of the following ratios must be maintained in the composition to achieve the tamper evidence: (a) a weight ratio of the gelling agent to the surfactant of at least between about 20:1 and about 30:1 (in other approaches, about 20:1 to about 25:1); (b) a weight ratio of the gelling agent to the sweetener of at least between about 5:1 to about 30:1 (in other approaches, about 20:1 to about 28:1 or, more preferably, about 20:1 to about 25:1); (c) a weight ratio of the surfactant to the sweetener of at least between about 0.1:1 to about 2:1 (in other approaches, about 0.8:1 to 1.5:1 or, more preferably, about 0.82 to about 1.2:1); and/or (d) a weight ratio of the flavor system to the surfactant of about 10:1 to about 25:1 (in other approaches, about 12:1 to about 24:1, or in yet other approaches, about 15:1 to about 22:1) to achieve tamper evidence of a two-part capsule when using flavors and/or sweeteners in the banding composition. As shown in the Examples below, when the banding compositions include the flavor component, if one or more of the relationships above are not maintained within these unique ranges, then the band for the capsules does not maintain tamper evidence.

In one approach or embodiment, the flavor banding compositions can be prepared as follows: (1) the gelling agent is first added to water containing the sweetener, surfactant, and any optional colorant to form a gelatin solution. The mixture is then kept in an oven at 55° C. to about 75° C. for a minimum of about 8 hours and up to about 24 hours. (2) A separate flavor dispersion is then prepared by adding the selected flavor to water and then stirred to make uniform dispersion. (3) The flavor dispersion prepared in step 2 may be stirred for about 1 to about 2 minutes (or as needed to form a dispersion) and placed back in the oven for about 1 to about 1.5 hours (optionally, the flavor dispersion may be combined with the gelatin solution prepared at step 1 before the oven heating). (4) After the completion of step 3, the flavored dispersion is removed from the oven and (if not already added to the gelatin solution of step 1) added to the gelatin solution prepared in step 1 and checked for viscosity. The viscosity of flavored gelatin dispersion can be adjusted with water, if required, to ensure a final viscosity in the range of about 120 to about 190 cps. Viscosity is measured herein using a Brookfield viscometer (or equivalent) with spindle #01 at 20 RPM and at a temperature of about 55° C. to about 65° C. The formed gelatin flavored dispersion can then be applied to a two-part capsule using any suitable banding process or equipment, such as discussed more below, a Hermetica capsule banding machine or equivalent.

In other approaches, a tamper-evident, two-part capsule having a flavored banding portion is described by this disclosure. The capsules may be filed with any suitable pharmaceutical compound and may be liquid, powders, or other suitable compounds. In embodiments, the capsule may have two capsule parts (i.e., a first capsule part, and a second capsule part), and a flavored banding portion encircling an interface between the two capsule parts effective to provide tamper evidence to the capsule pursuant to 21 C.F.R Section 211.132. In further approaches, the flavor banding portion is provided by any embodiment of the compositions described herein and may be at least a composition including (i) at least about 50 weight percent, on a dry basis, of a gelling agent, (ii) a surfactant, and (iii) a flavor system including both a sweetener and a flavor. As described above, the compositions herein include one or more of the following relationships to achieve tamper evidence: (a) a weight ratio of the gelling agent relative to the surfactant of about 20:1 to about 30:1 (in other approaches, about 20:1 to about 25:1); (b) a weight ratio of the gelling agent to the sweetener of about 5:1 to about 30:1 (in other approaches, about 20:1 to about 28:1 or, more preferably, about 20:1 to about 25:1): (c) a weight ratio of the surfactant to the sweetener is about 0.2:1 to about 2:1 (in other approaches, about 0.8:1 to 1.5:1 or, more preferably, about 0.82 to about 1.2:1); and/or (d) a weight ratio of the flavor system to the surfactant of about 10:1 to about 25:1 (in other approaches, about 12:1 to about 24:1, or in yet other approaches, about 15:1 to about 22:1). The compositions providing the flavor band herein may also include any further embodiment as described herein. The compositions may optionally include color, such as, on a dry basis, about 0 to about 4 weight percent color, about 0.8 to about 2 weight percent color or about 0.8 to about 1.2 weight percent of one or more suitable colorants.

The banded capsule is prepared as follows: The final dispersion obtained in step 4 above is transferred to the solution pot of Hermetica bander machine (or equivalent) and then applied on unbanded assembled, two-part capsules. After the application of wet band on the cap and body interface, the capsule may pass through the bander machine drying chamber. Generally, drying occurs at room temperature, such as about 20° C. to about 25° C. During the application of flavored banding dispersion on the capsules, the temperature of flavored gelatin dispersion was maintained in the range of about 40° C. to about 60° C.

In other approaches or embodiments, the capsule size is not particularly limited, and the capsule may have a size of 00 to 5, and the capsule may have about 2 mg to about 5 mg of the flavored banding portion, having the compositions as described herein, per capsule. In other approaches or embodiments, the capsule preferably has a size 4 and, at such size, the capsule includes about 2.5 to about 3.5 mg of the flavor banding portion per capsule, and in other approaches, about 2.8 to about 3.2 mg of the flavored banding portion, or preferably about 3 mg of the flavored banding portion per size 4 capsule.

In yet other approaches, the capsule is a gelatin capsule where at least the first and second capsule shell portions include or are comprised of gelatin. In this regard, it is preferred that the flavor banding composition include a composition effective to bond with the gelatin capsule and, in this regard, it is preferred that the gelling agent of the flavor banding composition is configured for the capsule. Thus, a gelatin capsule will use a flavor banding portion including gelatin as the gelling agent. In this regard, it is preferred that the flavor band composition provide at least about 1.5 mg to about 2 mg of gelatin in the flavor banding composition (in other approaches, about 1.5 mg to about 1.7 mg) for a size 4 capsule having about 2.8 to about 3.2 mg of a flavor band applied thereto, and preferably about 3 mg of a flavor bland applied thereto.

### Definitions

For purposes of this disclosure, any chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

Tamper evidence is defined by 21 C.F.R. Section 211.132.

A flavor is defined by 21 C.F.R. Section 101.22.

All weight percentages, unless stated otherwise, in the Examples and throughout this disclosure are reported on a dry basis without water.

Unless stated otherwise, solution viscosity in the Examples and throughout this disclosure is measured via a Brookfield viscometer (or equivalent) using spindle #1 at 20 rpm and at 55° C. to 65° C.

### EXAMPLES

A better understanding of the present disclosure and its many advantages may be clarified with the following examples. The following examples are illustrative and not limiting thereof in either scope or spirit. Those skilled in the art will readily understand that variations of the components, methods, steps, and devices described in these examples can be used. Unless noted otherwise or apparent from the context of discussion in the Examples below and throughout this disclosure, all percentages, ratios, and parts noted in this disclosure are by weight.

Banding compositions including a flavor system of a sweetener and a flavorant suitable for a gelatin two-part capsule were evaluated for tamper evidence as set forth in 21 C.F.R. Section 211.132. Inventive and comparative compositions are set forth in Table 1 below and show various unique relationships of the gelling agent, surfactant, sweetener, and/or flavor system that need to be maintained in order to achieve tamper evidence when the banding compositions also include about 1 to about 50 weight percent flavor on a dry basis (in other embodiments, about 30 to about 50 weight percent of a flavor on a dry basis). The compositions of this Example were used to provide a 3 mg band about the interface of a number 4 size two-part gelatin capsule.

The banded capsules were prepared from a composition including gelatin as the gelling agent, polysorbate 80 as the surfactant, color, sucralose as the sweetener, and cool mint flavor as the flavorant as set forth in the compositions of Table 1 below. The flavor compositions were prepared as follows: (1) The gelatin was added to about 2.6 kg of purified water containing the sucralose, the polysorbate 80, and the color. The mixture was kept in hot air oven overnight at 65° C. (generally about 55° C. to about 75° C.) for a minimum of 8 hours to 24 hours. (2) A separate flavor dispersion was prepared by adding the flavor to about 1.5 kg purified water and then stirred to make uniform dispersion. (3) The flavor dispersion prepared in step 2 was then added to gelatin solution of step 1 and was stirred for 1 to 2 minutes and placed back in the oven at 65° C. (generally about 55° C. to about 75° C.) for 1 to 1.5 hours. (4) After the completion of step 3, the dispersion was removed from the oven and checked for viscosity. The viscosity of flavored gelatin dispersion was adjusted with water, if required, to ensure the final viscosity in the range of 120 to 190 cps. (5) The final dispersion obtained in step 4 was then transferred to the solution pot of Hermetica 100 bander and then applied on the unbanded capsules and dried at room temperature. (6) During the application of flavored banding dispersion on the capsules, the temperature of each flavored gelatin dispersion was maintained in the range of about 55° C. to about 75° C.

TABLE 1
Banding Compositions¹

| | Compare | Compare | Compare | Inventive | Inventive |
|---|---|---|---|---|---|
| Ingredient | 1 | 2 | 3 | 1 | 2 |
| Gelatin | 95.9% | 28.1% | 43.3% | 50.0% | 56.7% |
| Polysorbate 80 | 4.1% | 1.2% | 1.9% | 2.1% | 2.4% |
| Color | 0.1% | 1.0% | 1.0% | 1.0% | 1.0% |
| Sucralose | — | 3.0% | 2.3% | 2.3% | 2.3% |
| Flavor | — | 66.7% | 51.5% | 44.7% | 37.7% |
| Flavor system | — | 69.7% | 53.8% | 47.0% | 40% |
| (e.g. sucralose | | | | | |
| and flavor) | | | | | |
| Sweetener in | — | 4.3% | 4.3% | 5.0% | 5.8% |
| Flavor System | | | | | |
| Flavor in | — | 95.7% | 95.7% | 95.0% | 94.2% |
| Flavor | | | | | |
| System | | | | | |
| Ratio | 23.4:1 | 23.4:1 | 22.8:1 | 23.8:1 | 23.6:1 |
| gelling agent | | | | | |
| to surfactant | | | | | |
| Ratio | — | 9.4:1 | 18.8:1 | 21.7:1 | 24.7:1 |
| gelling agent | | | | | |
| to sweetener | | | | | |
| Ratio | — | 0.4:1 | 0.8:1 | 0.9:1 | 1.0:1 |
| surfactant | | | | | |
| to sweetener | | | | | |
| Ratio flavor | — | 58.1:1 | 28.3:1 | 22.4:1 | 16.7:1 |
| system | | | | | |
| to surfactant | | | | | |
| Tamper | Yes | No | No | Yes | Yes |
| Evident | | | | | |
| Seal | | | | | |

¹Compositions reported on dry basis and water used during preparation was removed during the drying process.

As shown in Table 1 above, when the flavor system including the flavor and sweetener is added to the banding composition, tamper evidence can only be obtained when the composition includes at least 50 weight percent gelatin and/or when various noted relationships of gelatin, surfactant, sweetener, and/or flavor system are maintained within the discovered relationships discussed above and when the compositions include the flavors.

It is noted that, as used in this specification and the appended claims, the singular forms “a,” “an,” and “the,” include plural referents unless expressly and unequivocally limited to one referent. Thus, for example, reference to “an antioxidant” includes two or more different antioxidants. As used herein, the term “include” and its grammatical variants are intended to be non-limiting, such that recitation of items in a list is not to the exclusion of other like items that can be substituted or added to the listed items

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is to be understood that each component, compound, substituent or parameter disclosed herein is to be interpreted as being disclosed for use alone or in combination with one or more of each and every other component, compound, substituent or parameter disclosed herein.

It is further understood that each range disclosed herein is to be interpreted as a disclosure of each specific value within the disclosed range that has the same number of significant digits. Thus, for example, a range from 1 to 4 is to be interpreted as an express disclosure of the values 1, 2, 3 and 4 as well as any range of such values.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range. That is, it is also further understood that any range between the endpoint values within the broad range is also discussed herein. Thus, a range from 1 to 4 also means a range from 1 to 3, 1 to 2, 2 to 4, 2 to 3, and so forth.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

What is claimed is:

1. A composition suitable for a flavored banding portion of a tamper-evident, two-part capsule, the composition comprising:
at least about 50 weight percent, on a dry basis, of a gelling agent;
a surfactant;
a flavor system including a sweetener and a flavor;
wherein a weight ratio of the flavor system to the surfactant is about 10:1 to about 25:1; and
wherein the composition is effective to provide tamper evidence to a two-part capsule that complies with the requirements of 21 C.F.R. Section 211.132.

2. The composition suitable for a flavored banding portion of a tamper-evidence two-part capsule of claim 1, wherein a weight ratio of the gelling agent relative to the surfactant is about 20:1 to about 30:1, a weight ratio of the gelling agent to the sweetener is about 5:1 to about 30:1, and/or a weight ratio of the surfactant to the sweetener is about 0.2:1 to about 2:1.

3. The composition suitable for a flavored banding portion of a tamper-evident, two-part capsule of claim 1, wherein the gelling agent is selected form the group consisting of gelatin, locust bean gum, agar, guar, carrageenan, konjac, pectin, or combinations thereof.

4. The composition suitable for a flavored banding portion of a tamper-evident, two-part capsule of claim 1, wherein the composition includes, on a dry basis, about 50 to about 95 weight percent of the gelling agent.

5. The composition suitable for a flavored banding portion of a tamper-evident, two-part capsule of claim 1, wherein the surfactant is selected from the group consisting of polyoxyethylene sorbitan monooleate, sorbitan monostearate, sorbitan stearate, or combinations thereof.

6. The composition suitable for a flavored banding portion of a tamper-evident, two-part capsule of claim 5, wherein the composition includes, on a dry basis, about 1 to about 4 weight percent of the surfactant.

7. The composition suitable for a flavored banding portion of a tamper-evident, two-part capsule of claim 1, wherein the sweetener is a natural or non-nutritive sweetener selected from the group consisting of sugar, sucrose, glucose, sucralose, aspartame, acesulfame k, saccharin, stevia, or combinations thereof.

8. The composition suitable for a flavored banding portion of a tamper-evident, two-part capsule of claim 7, wherein the composition includes, on a dry basis, about 1 to about 50 weight percent of the sweetener.

9. The composition suitable for a flavored banding portion of a tamper-evident, two-part capsule of claim 1, wherein the weight ratio of the gelling agent to the sweetener is about 20:1 to about 25:1 and the weight ratio of the surfactant to the sweetener is about 0.8:1 to about 1.2:1.

10. The composition suitable for a flavored banding portion of a tamper-evident, two-part capsule of claim 1, wherein the gelling agent is gelatin, the surfactant is polyoxyethylene sorbitan monooleate, and the sweetener is sucralose.

11. The composition suitable for a flavored banding portion of a tamper-evident, two-part capsule of claim 1, wherein the composition includes, on a dry basis, up to about 50 weight percent of the flavor.

12. A tamper-evident, two-part capsule having a flavored banding portion, the capsule comprising:
a capsule having two capsule parts;
a flavored banding portion encircling an interface between the two capsule parts effective to provide tamper evidence to the capsule;
wherein the flavored banding portion is provided by a composition including (i) at least about 50 weight percent, on a dry basis, of a gelling agent, (ii) a surfactant, and (iii) a flavor system including a sweetener and a flavor, wherein a weight ratio of the flavor system to the surfactant is about 10:1 to about 25:1; and
wherein the tamper-evident, two-part capsule is tamper evident pursuant to the requirements of 21 C.F.R. Section 211.132.

13. The tamper-evident, two-part capsule of claim 12, wherein a weight ratio of the gelling agent relative to the surfactant is about 20:1 to about 30:1, a weight ratio of the gelling agent to the sweetener is about 5:1 to about 30:1, and/or a weight ratio of the surfactant to the sweetener is about 0.2:1 to about 2:1.

14. The tamper-evident, two-part capsule of claim 12, wherein the gelling agent is selected form the group consisting of gelatin, locust bean gum, agar, guar, carrageenan, konjac, pectin, or combinations thereof.

15. The tamper-evident, two-part capsule of claim 12, wherein the composition includes, on a dry basis, about 50 to about 95 weight percent of the gelling agent.

16. The tamper-evident, two-part capsule of claim 12, wherein the composition includes, on a dry basis, about 50 to about 60 weight percent of the gelling agent.

17. The tamper-evident, two-part capsule of claim 12, wherein the surfactant is selected from the group consisting of polyoxyethylene sorbitan monooleate, sorbitan monostearate, sorbitan stearate, or combinations thereof.

18. The tamper-evident, two-part capsule of claim 12, wherein the composition includes, on a dry basis, about 1 to about 4 weight percent of the surfactant.

19. The tamper-evident, two-part capsule of claim 12, wherein the sweetener is a natural or non-nutritive sweetener selected from the group consisting of sugar, sucrose, glucose, sucralose, aspartame, acesulfame k, saccharin, stevia, or combinations thereof.

20. The tamper-evident, two-part capsule of claim 12, wherein the composition includes, on a dry basis, about 1 to about 50 weight percent of the sweetener.

21. The tamper-evident, two-part capsule of claim 12, wherein the gelling agent is gelatin, the surfactant is polyoxyethylene sorbitan monooleate, and the sweetener is sucralose.

22. The tamper-evident, two-part capsule of claim 12, wherein the composition includes, on a dry basis, up to about 50 weight percent of the flavor.

23. The tamper-evident, two-part capsule of claim 12, wherein the capsule has a size of 00 to 5 and about 2 mg to about 5 mg of the flavored banding portion per capsule.

24. The tamper-evident, two-part capsule of claim 12, wherein the capsule has a size 4 and includes about 2.8 to about 3.2 mg of the flavored banding portion per capsule.

25. The tamper-evident, two-part capsule of claim 12, wherein the capsule is a gelatin capsule and the gelling agent is gelatin.
